(19) United States
(12) Patent Application Publication
(10) Pub. No.: US 2021/0022894 A1
(43) Pub. Date: Jan. 28, 2021

(54) System And Method For Percutaneous Removal Of Transcatheter Heart Valves

(71) Applicants: The Regents of the University of California, Oakland, CA (US); The United States Government Represented by the Department of Veterans Affairs, Washington, DC (US)

(72) Inventors: Elaine Tseng, Oakland, CA (US); John Ashley, Oakland, CA (US); Liang Ge, Oakland, CA (US); Miles Alexander, Oakland, CA (US); Brett Follmer, Oakland, CA (US); Brenna Lord, Oakland, CA (US)

(21) Appl. No.: 17/043,965

(22) PCT Filed: Apr. 4, 2019
(86) PCT No.: PCT/US2019/025447

§ 371 Date: Sep. 30, 2020

Related U.S. Application Data
(60) Provisional application No. 62/651,568, filed on Apr. 2, 2018.

(51) Int. Cl. A61F 2/95; A61F 2/24

(52) U.S. Cl. CPC A61F 2/95; A61F 2/24; A61F 2002/9583; A61F 2002/9511; A61F 2002/9528

(57) ABSTRACT

The disclosure features a method for removing a transcatheter heart valve from a heart in a patient by providing a removal device that deploys one or more transcatheter engagement elements to engage the transcatheter heart valve.

### CROSS-REFERENCE TO RELATED APPLICATION(S)

This application claims priority to International PCT Application No. PCT/US19/25447, filed on Apr. 2, 2019, which claims the benefit under 35 U.S.C. § 119(e) to U.S. Provisional Application 62/651,568, filed Apr. 2, 2018 and entitled “System And Method For Percutaneous Removal Of Transcatheter Heart Valves,” which is hereby incorporated herein by reference in its entirety.

### BACKGROUND OF THE INVENTION

An unmet clinical need is emerging for percutaneously removing and replacing transcatheter heart valves for the surgical patient population. Until 2016, transcatheter heart valve patients had been high-risk surgical or inoperable patients with an average age of 80. As such the majority of these patients have not outlived their transcatheter heart valve's durability which has been reported to be approximately 8 years. Surgical patients are younger and healthier; and current transcatheter heart valves will undoubtedly fail within those patients' lifetime. In 2016, transcatheter aortic valve replacement (TAVR) was FDA approved for the intermediate-risk population after 2-year clinical studies of TAVR showed equivalent to superior outcomes compared to surgical aortic valve replacement (SAVR) in this patient population. Younger, low-risk surgical patients are also demanding a transcatheter approach due to the lower morbidity as compared to SAVR, which requires open heart surgery with the patient on cardiopulmonary bypass. Randomized trials are currently underway to support TAVR for low risk patients. Typically the TAVR patient has a 2 day hospital stay as compared to the SAVR patient that stays for 5-7 days. Currently, there is no device that can remove a failed transcatheter heart valves using a transcatheter approach. Also, transcatheter mitral valve replacement (TMVR) is a burgeoning market predicted to rival TAVR. TMVR will ultimately have similar durability concerns and a need for percutaneous replacement strategies. As well, pediatric valve disease patients often require multiple valve replacements due to growth of the child. A percutaneous valve replacement device would also benefit these patients. A device for percutaneously removing transcatheter valves could be used for surgical adult and pediatric patients with either aortic or mitral valve disease who prefer a percutaneous option not only for their first but also for their subsequent valve replacements.

### BRIEF SUMMARY OF THE INVENTION

In one aspect, the disclosure features a method for removing a transcatheter heart valve from a heart in a patient, the method comprising: providing a removal device comprising a control handle and a flexible shaft, the flexible shaft having a proximal end coupled to the control handle, a lumen, and a distal opening; positioning the distal end of the flexible shaft such that the distal opening is disposed next to the transcatheter heart valve in the heart; deploying one or more transcatheter engagement elements from the lumen through the distal opening of the flexible shaft such that the engagement elements engage the transcatheter heart valve; and retracting the one or more engagement elements such that the transcatheter heart valve is removed.

In some embodiments of this aspect, the step of retracting the one or more engagement elements pulls the transcatheter heart valve through the distal end into the lumen of the flexible shaft.

In some embodiments, the distal end of the flexible shaft includes a funnel which reduces the diameter of the transcatheter heart valve when the one or more engagement elements pulls the transcatheter heart valve through the distal end into the lumen of the flexible shaft.

In some embodiments, the engagement element comprises an elongate element that enables the engagement element to reach the transcatheter heart valve and a jaw that grips the transcatheter heart valve.

In some embodiments, the engagement element comprises an elongate element that enables the engagement element to reach the transcatheter heart valve and a locking gate that grips the transcatheter heart valve. In particular embodiments, the locking gate may comprise a stop, a gate, and an articulating hinge.

In some embodiments, the engagement element comprises an engagement capture loop, a loop shaft, and an engagement capture wire. In particular embodiments, the engagement capture loop is advanced on one side of the transcatheter heart valve by extending the loop shaft, wherein the engagement capture wire is advanced on the other side of the transcatheter heart valve and inserted through the engagement capture loop, thereby capturing the transcatheter heart valve between the engagement capture loop and the engagement capture wire.

In some embodiments, the engagement element comprises an engagement coil. The engagement coil may be tapered. The engagement coil may comprise a variable pitch.

In some embodiments, the engagement element comprises a suture, a suture thrower, a suture grasping jaw, a jaw pivot, and a suture support shaft. In particular embodiments, the suture thrower advances the suture from one side of the transcatheter heart valve to the suture grasping jaw on the other side of the transcatheter heart valve. Further, in some embodiments, the jaw pivot enables the suture grasping jaw to open and close.

In some embodiments, the engagement element comprises a pre-shaped hook and a hook containment tube. In particular embodiments, the hook containment tube advances over the distal end of the pre-shaped hook to lock the pre-shaped hook to the transcatheter heart valve once the pre-shaped hook advances through the transcatheter heart valve.

In some embodiments, the transcatheter heart valve comprises one or more wire docking extensions and the engagement element comprises a central shaft, one or more engagement wires, and a wire hub. The central shaft, the engagement wires, and the wire hub may be rotated together to engage the wire docking extensions.

In other embodiments, the engagement element comprises an attachment tube, one or more attachment rings, and a tube collapsing element, wherein the attachment tube is connected to the transcatheter heart valve by the attachment rings.

In other embodiments, the transcatheter heart valve comprises one or more clips or rings, one or more extensions, and a central dock, wherein the clips or rings are attached to the central dock through the extensions.

In some embodiments, the removal device further comprises a compression balloon.

In another aspect, the disclosure features a method for removing a transcatheter heart valve from a heart in a patient, the method comprising: providing a removal device comprising a control handle and a flexible shaft, the flexible shaft having a proximal end coupled to the control handle, a lumen, and a distal opening; positioning the distal end of the flexible shaft such that the distal opening is disposed next to the transcatheter heart valve in the heart; deploying a compression balloon from the lumen through the distal opening of the flexible shaft such that the compression balloon engages the transcatheter heart valve; and retracting the one or more engagement elements such that the transcatheter heart valve is removed.

### BRIEF DESCRIPTION OF THE DRAWINGS

FIG. 1A is a perspective view of one embodiment of the current invention.

FIG. 1B is a detailed view of the distal end of the embodiment of the current invention shown in FIG. 1A.

FIG. 2A is a perspective view of the embodiment of the current invention shown in FIG. 1A with the retriever deployed.

FIG. 2B is a detailed view of the distal end of the embodiment of the current invention shown in FIG. 2A.

FIG. 3 is a perspective view of the distal end of the embodiment of the current invention shown in FIG. 1A with the retriever partially retracted.

FIG. 4 is a perspective view of the distal end of the embodiment of the current invention shown in FIG. 1A with the retriever substantially retracted.

FIG. 5A is a perspective view of the distal end of an alternate embodiment of the current invention.

FIG. 5B is a detailed view of the distal end of the embodiment of the current invention shown in FIG. 5A.

FIG. 6A is a perspective view of the distal end of another alternate embodiment of the current invention.

FIG. 6B is a detailed view of the distal end of the embodiment of the current invention shown in FIG. 6A.

FIG. 7 is a perspective view of the distal end of another alternate embodiment of the current invention.

FIG. 8 is a perspective view of the distal end of another alternate embodiment of the current invention.

FIG. 9 is a perspective view of the distal end of another alternate embodiment of the current invention.

FIG. 10A is a perspective view of the distal end of another alternate embodiment of the current invention.

FIG. 10B is a perspective view of the distal end of the embodiment of the current invention shown in FIG. 10A with the retriever partially expanded.

FIG. 11A is a perspective view of the distal end of another alternate embodiment of the current invention.

FIG. 11B is a detailed view of the distal end of the embodiment of the current invention shown in FIG. 11A.

FIG. 12A is a perspective view of the distal end of another alternate embodiment of the current invention and an alternate modification to a retrieval target.

FIG. 12B is a detailed view of the distal end of the embodiment of the current invention shown in FIG. 12A.

FIG. 13 is a perspective view of a modification to a retrieval target.

FIG. 14 is a perspective view of the distal end of another alternate embodiment of the current invention.

### DETAILED DESCRIPTION OF THE INVENTION

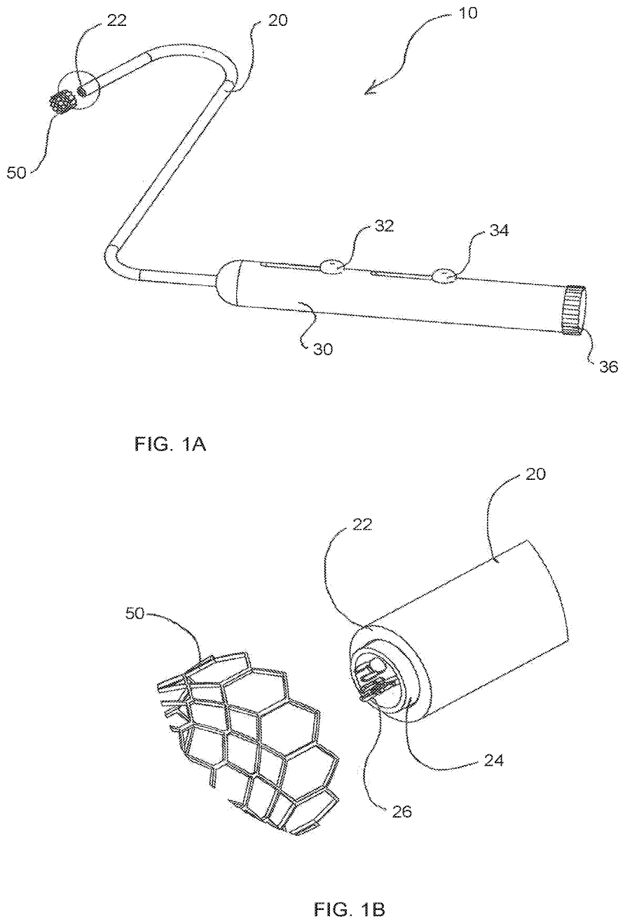

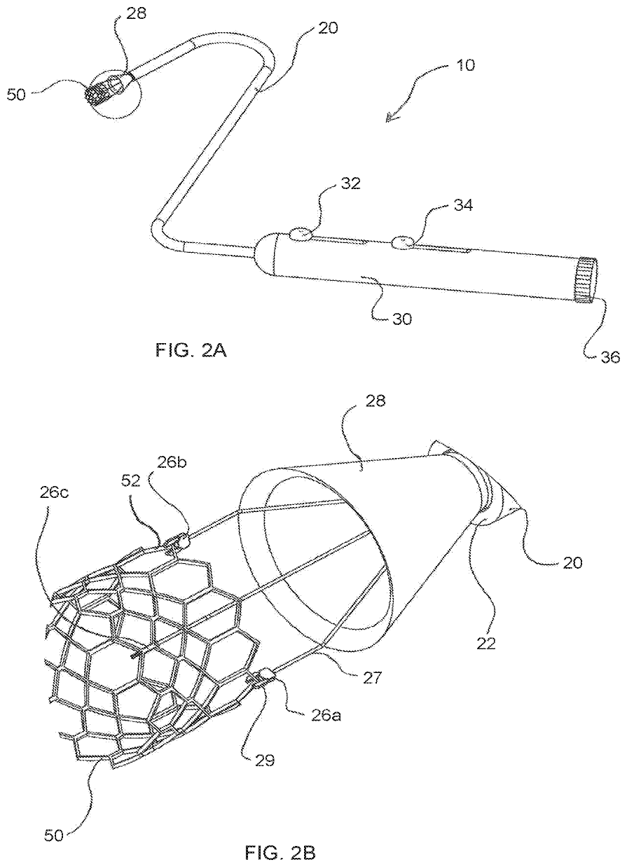

Referring to FIGS. 1A, 1B, 2A, and 2B, a valve removal device 10 for removal of a transcatheter valve 50 is shown. The valve removal device 10 is comprised of a flexible shaft 20, with a control handle 30 at one end of the flexible shaft 20, and a distal opening 22 at the other end of the flexible shaft 20. The flexible shaft 20 allows insertion of the valve removal device 10 into the patient's vasculature such as, but not limited to, the femoral artery, the femoral vein, the jugular vein, the carotid artery, axillary artery, the left ventricular apex, and the like, which allows access to the transcatheter heart valve.

The flexible shaft 20 can be comprised of a polymer jacket on top of a braid of metallic wires over a central core that is comprised of a low-friction material with one of more lumens. The polymer jacket can be made of Pebax® (Arkema, Colombes, France) or a similar thermoplastic polymer. The metal wires can be a 303 stainless steel, a nickel or silver coated copper, or similar metal. The low-friction central core can be made of a fluoropolymer such as PTFE (Polytetrafluoroethylene) or it can by a thermoplastic with a lubricious coating such as Parylene. The flexible shaft 20 not only guides the rest of the valve removal device 10 through the patient's vasculature to the implanted targeted transcatheter valve 50, but also provides the supporting strength to impart the forces required to remove the valve 50. Once positioned in the vasculature, the flexible shaft 20 can be advanced to the location of the targeted transcatheter valve 50 in the patient's heart.

The articulation control knob 36 may be manipulated to allow the flexible shaft 20 to be curved to allow navigation in tortuous vessels. The distal opening 22 of the flexible shaft 20 provides for the extension and deployment of transcatheter valve 50 engagement elements 26, and transcatheter valve 50 diameter reduction elements 28 that are contained within the flexible shaft 20 during vascular access and advancement. Once the valve removal device 10 is advanced adjacent the transcatheter valve 50, one or more engagement elements 26a, 26b, and 26c are extended out of the distal opening 22 by means of an engagement control lever 32 on the control handle 30. There may be one, two, three, four, six, eight, nine, twelve or another number of engagement elements 26a, 26b, and 26c.

The one or more engagement elements 26a, 26b, and 26c are connected to the engagement control lever 32 by means of one or more elongate elements 27 that articulates through the flexible shaft 20. The elongate elements 27 can be made of a single metallic wire (stainless steel, nickel titanium, or the like) or polymer fiber or a metallic, polymer or combination cable comprised of multiple wires and/or fibers. The one or more engagement elements 26a, 26b, and 26c are extended until they connect with a section 52 of the transcatheter valve 50. In the embodiment shown in FIGS. 1A, 1B, 2A, and 2B, the one or more engagement elements 26a, 26b, and 26c have jaws 29 which grip a strut 52 of the transcatheter valve 50. The jaws 29 are configured to be pushed onto the strut 52 with relatively low force, but they require a relatively high force to be pulled off of the strut 52 allowing the engagement element to generate the force required to remove the transcatheter valve 50 from the patient's heart.

The one or more engagement elements 26a, 26b, and 26c can be advanced to grip one or more struts 52 sequentially one at a time or simultaneously as a group. There are many different possible jaw configurations to achieve this, including but not limited to, a chamber or radius on the distal face that aligns the jaws 29 with the strut 52 and expands the jaws 29 slightly to push over the strut 52, a spring that allows the jaws 29 to separate enough to pass over the strut 52 but then pulls the jaws 29 together again to hold onto the strut 52, a toothed edge on the jaws 29 that angles towards the catheter handle 30, a ratchet mechanism that allows the jaws 29 to open but then locks the jaws 29 closed, and the like. Rather than the jaws 29 articulating as they are pushed over the strut 52, the jaws 29 can be formed from a single piece of metal or polymer that has the elasticity to expand over the strut 52 and then close around it. Alternately, the jaws 29 can be actively articulated open and closed by translating an actuation mechanism that runs through the flexible shaft 22 to the articulation control knob 36 on the control handle 30.

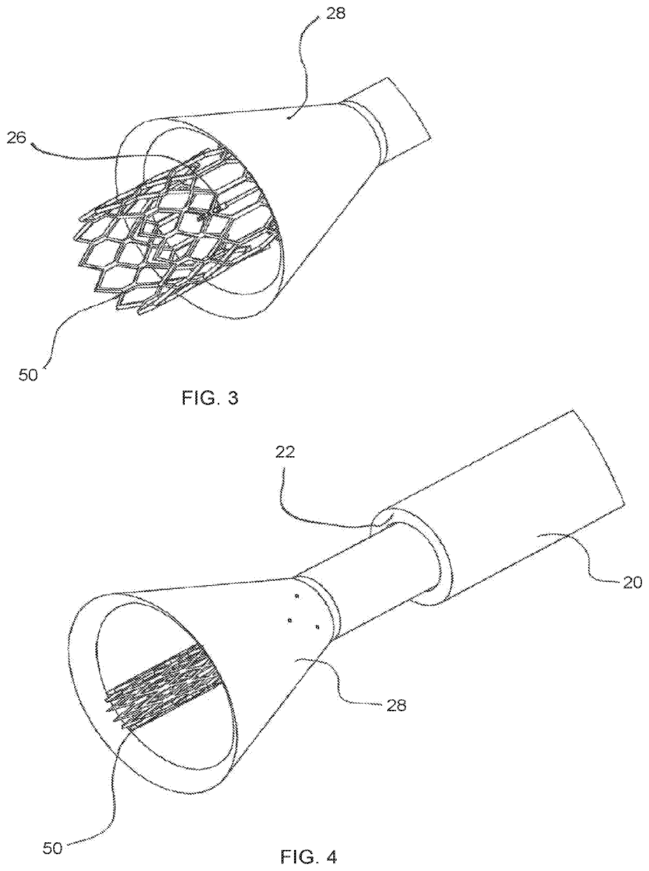

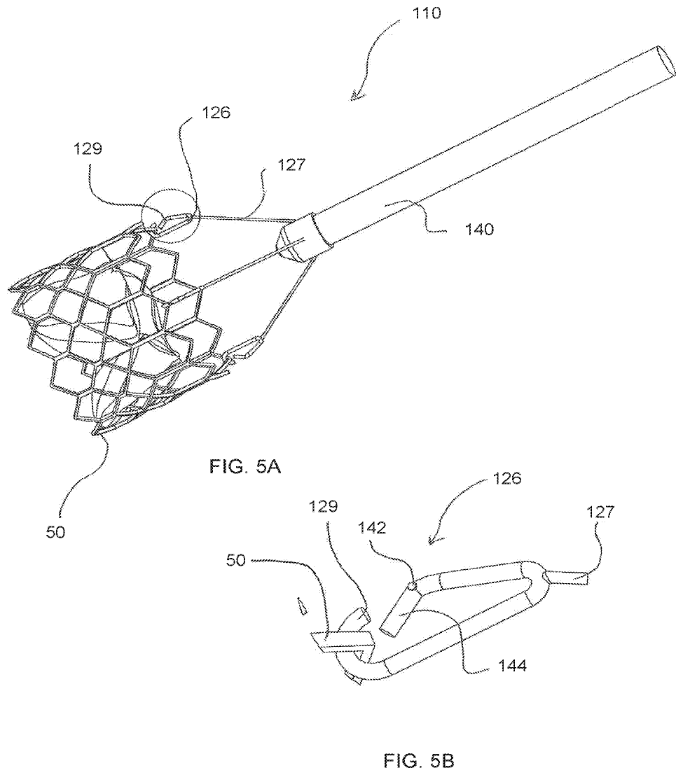

Once the one or more jaws 29 have engaged the transcatheter valve 50, the transcatheter valve 50 can be removed from its location in the patient's heart by retracting the engagement elements 26a, 26b, and 26c by means of the engagement control lever 32 or by translating the entire removal device 10. In addition to retracting the transcatheter valve 50 from its location, it is desirable to reduce the diameter of the transcatheter valve 50 to enable transporting the valve 50 through the vasculature and out of the patient. The diameter reduction element 28 can be extended out of the distal opening 22 by means of the diameter control lever 34 on the control handle 30. As shown in FIGS. 3 and 4, the diameter reduction element 28 forms a funnel which reduces the diameter of the transcatheter valve 50 when the engagement element 26 pulls the valve 50 towards the distal opening 22. The diameter control element 28 can be configured from a slotted and/or folded sheet of elastic metal or polymer that forms the funnel shape as the diameter control element 28 is extended out of the distal opening 22. Alternately, the distal opening 22 can itself by configured with an internal chamber or radius such that retracting the engagement elements 26a, 26b, and 26c back into the distal opening 22 reduces the diameter of the transcatheter valve 50 sufficiently for the transcatheter valve 50 to be retracted partially or fully into the flexible shaft 20 and removed from the patient.

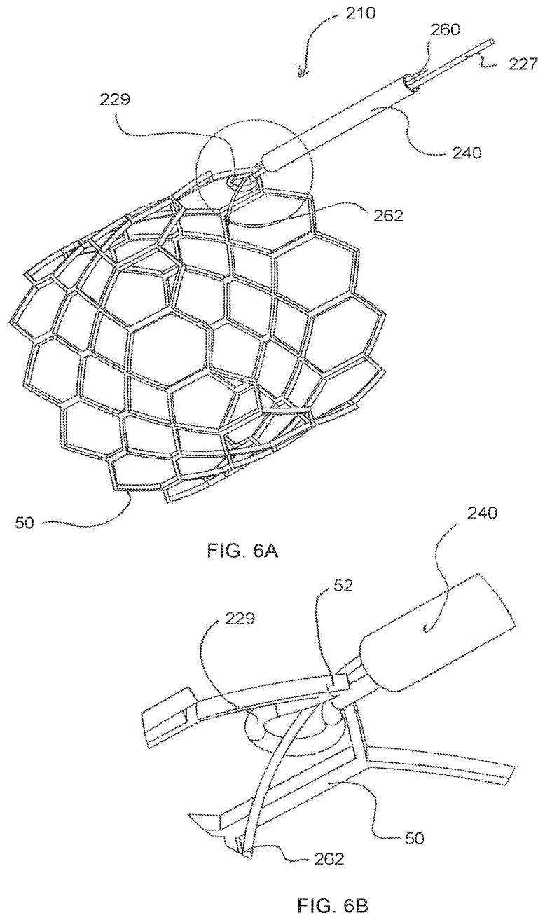

In an alternate valve removal device 110 as shown in FIGS. 5A and 5B, the one or more engagement elements 126 are configured as a one way locking gate comprised of a stop 129, a gate 144, and an articulating hinge 142. The engagement elements 126 are extended towards the transcatheter valve individually or as a group 50 until the valve strut 52 pushes open and past the gate 144 which closes and captures the valve 50. As with the previous embodiment shown in FIGS. 1A, 1B, 2A, 2B, 3, and 4, the engagement elements 126 in this embodiment can be passively activated, or an additional control element can be added to actively open and close the gate 144 with the articulation control knob 36. The engagement elements 126 can also be held in place using tension without the need for a gate.

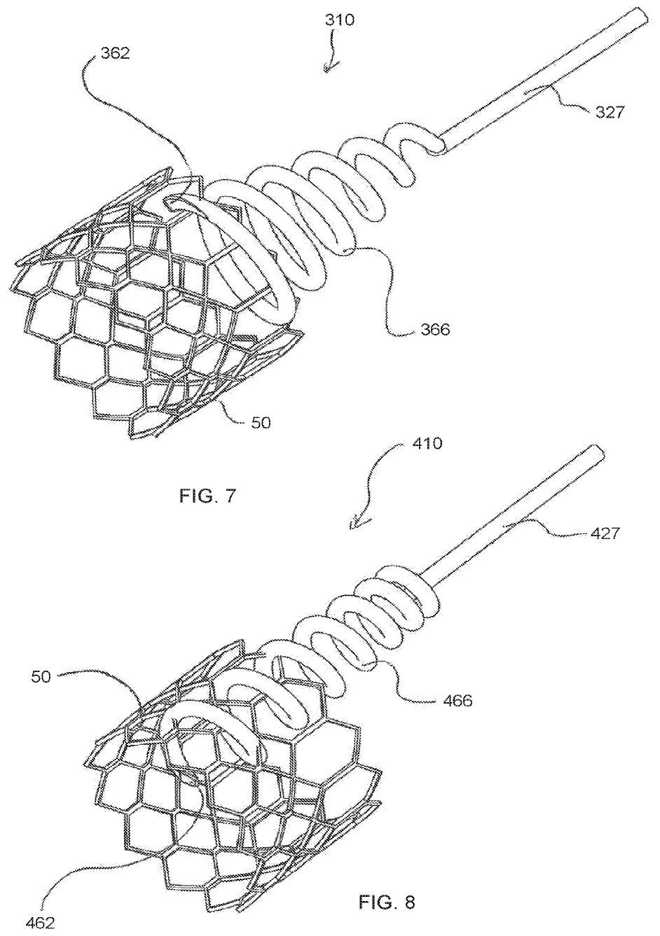

In FIGS. 6A and 6B, another alternate embodiment of the valve removal device 210 is shown where an engagement capture loop 229 is advanced on one side of a valve strut 52 by advancing the loop shaft 227 and the end 262 of an engagement capture wire 260 is advanced on the other side of a valve strut 52 and through the engagement capture loop thereby capturing the strut 52 between the engagement capture loop 229 and the engagement capture wire 260 for removal of the transcatheter valve 50. A guide tube 240 is provided to ensure proper alignment between the engagement capture loop 229 and the engagement capture wire 260. Although only a single set of engagement capture loop 229, engagement capture wire 260, and guide tube 240 are shown in these figures any number of sets of these elements can be used to engage the transcatheter valve 50 at multiple locations to facilitate removal.

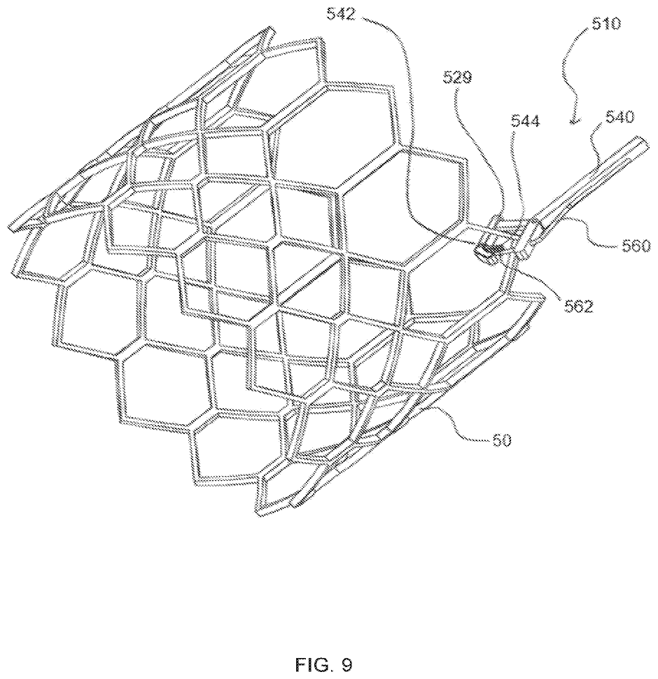

In FIG. 7, another alternate embodiment of the valve removal device 310 is shown where the distal end 362 of an engagement coil 366 is advanced through the valve frame 50 by rotating the coil shaft 327. The end 362 can be advanced from the inside diameter of the valve frame 50 to the outside diameter and back again to the inside diameter as shown in FIG. 7 or it can be first engaged around the outside diameter to the inside diameter of the valve frame 50. The engagement coil 366 can be tapered as shown such that further rotation of the coil shaft 327 will advance the more proximal smaller diameter coils around the valve frame 50 and thereby reduce the diameter of the valve frame to further facilitate removal. Once the engagement coil is fully advanced around the valve frame 50, the coil shaft 327 can be retracted to remove the valve frame 50 from its implanted location and pull the valve frame 50 into a diameter reduction element 28 or if already sufficiently reduced in diameter by the tapered engagement coil 366 directly into the flexible shaft 20. The diameter of the engagement coil 366 in FIG. 7 is sized to engage only a portion of the diameter of the valve frame 50, but alternate diameters of the engagement coil 366 are possible that engage a larger portion of diameter of the valve frame 50, a smaller portion of the diameter of the valve frame 50, or the entire outer diameter of the valve frame 50.

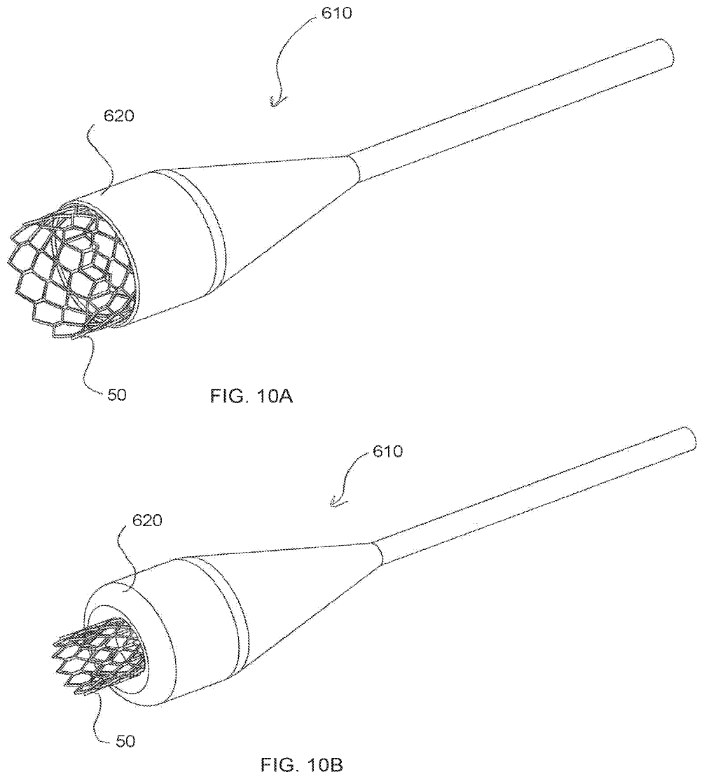

In FIG. 8, an alternate embodiment of the valve removal device 410 is shown where the distal end 462 of an engagement coil 466 is advanced through the valve frame 50 by rotating the coil shaft 427. In this embodiment, the engagement coil 466 has a constant diameter but a variable pitch. The variable pitch of the coils of the engagement coil 466 allow the distal end 462 to easily engage the valve frame 50 due to the open pitch of coils near the distal end 462. Further rotation of the coil shaft 427 advances tighter pitched coils around the valve frame 50 which provide a more secure attachment of the engagement coil 466 to the valve frame 50. A secure attachment is desirable so that sufficient force is provided between the engagement coil 466 and the valve frame 50 to enable the coil shaft 427 to be withdrawn with sufficient force to retract the valve frame 50 from its implanted location without separation of the engagement coil 466 from the valve frame 50. As with previous embodiments, both the valve removal device 310 shown in FIG. 7 and the valve removal device 410 shown in FIG. 8 can be comprised of a single engagement coil 366 or 466, or they can be comprised of two or more engagement coils 366 or 466 in order to further facilitate percutaneous removal of the valve frame 50 from its implant location.

The valve frame 50 can be withdrawn from the implantation site by tensioning the coil shaft 427 as described herein, but it can also be withdrawn by a combination of torqueing as well as tensioning one or more coil shafts 427. Torque applied by the coil shaft 427 to the valve frame 50 will twist the valve frame into a smaller diameter which will help to facilitate removal. Torque can be applied by any one of the valve removal devices 10 described herein. Torque can also be applied selectively between more than one valve frame 50 engagement elements to twist the valve frame 50 and reduce its diameter.

Torque can be applied to the valve frame 50 to reduce its diameter with a coil shaft 427 as shown in FIG. 8, but it can also be applied with a simple double pronged fork like element where each prong is positioned on either side of the valve frame 50 and the double pronged fork element is rotated. In addition, such a double pronged fork element can be positioned through the valve frame 50 (perpendicular to the position on either side) and then rotated to pull the valve frame and reduce its length and pull it from its implanted position.

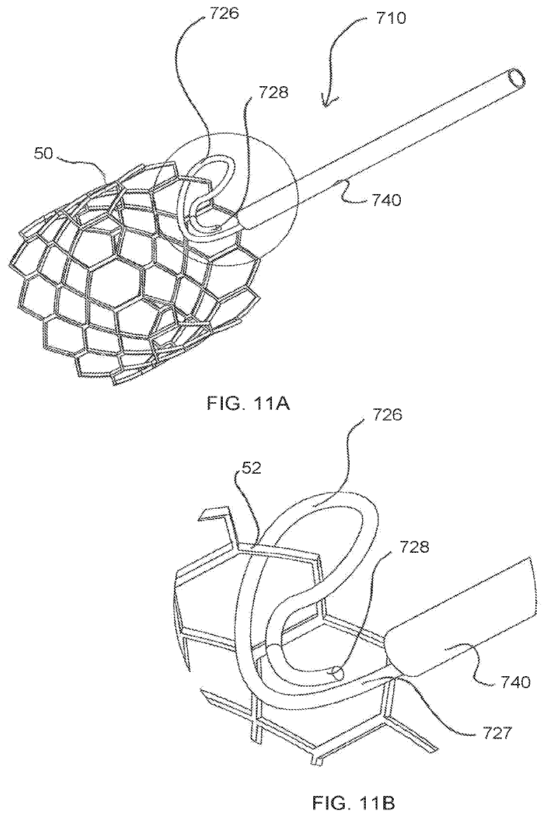

In FIG. 9, an alternate embodiment of the valve removal device 510 is shown comprised of a suture thrower 562, a suture grasping jaw 544 and a suture 560. The suture thrower 562 advances the suture 560 from one side of the valve frame 50 to the suture grasping jaw 544 on the other side of the valve frame 50. The suture grasping jaw 544 can pivot about the jaw pivot 542 to close on and grasp the suture 560. Once the suture grasping jaw 544 has grasped the suture, the suture support shaft 540 can be retracted and the valve frame 50 can be drawn away from the implant location and into a diameter reduction element 28 then into the flexible shaft 20. Alternately, the grasped suture can be drawn into the flexible shaft 20 and out of the end of the control handle 30. Then a second suture 560 can be engaged by the suture thrower 562 and passed through a different section of the valve frame 50 to the suture grasping jaws 544 and then pulled back through the flexible shaft 20 and out of the end of the control handle 30. This process can be repeated as many times as necessary in order to provide sufficient points of contact by engaged sutures 560 to the valve frame 50 as are needed to remove the valve frame 50 from its implanted location by pulling on the engaged sutures 560.

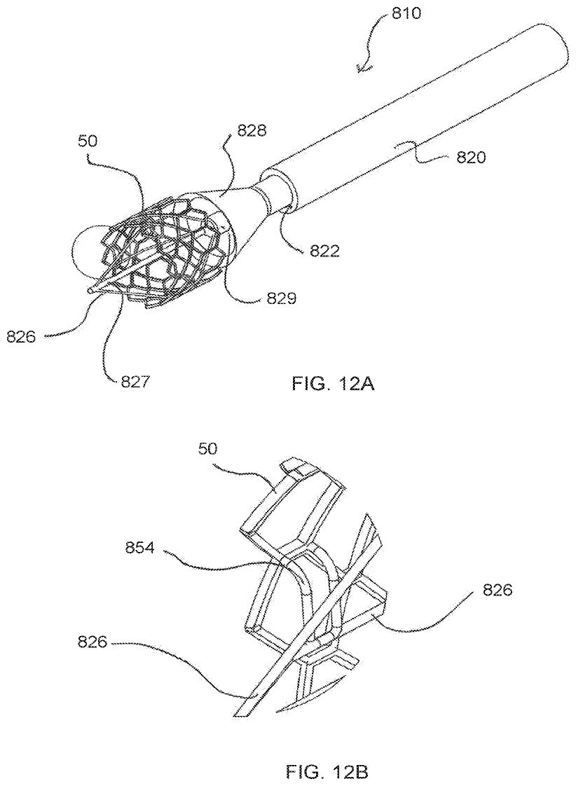

In FIGS. 10A and 10B, an alternate embodiment of the valve removal device 610 is shown comprised of a compression balloon 620. The compression balloon 620 is configured such that it has an inner diameter that is larger than the outer diameter of the valve frame 50 when it is deployed from the flexible shaft 20. The outer diameter of the compression balloon 620 is configured to not expand beyond a specific diameter consisted with a diameter less than that of the patient's inner aorta diameter. The compression balloon 620 can be advanced over the valve frame 50 as shown in FIG. 10A and then expanded to compress the valve frame 50 as shown in FIG. 10B. Once the valve frame is compressed, the compression balloon 620 can be partially deflated such that the valve frame 50 can be drawn with the compression balloon 620 into the flexible shaft 20. The compression balloon 620 can also be combined with any of the other described embodiments where the compression balloon 620 compresses the valve frame 50 and the other embodiments withdraw the valve frame 50 into the flexible shaft 20, or into a further diameter reduction element 28 and then into the flexible shaft 20. It is further provided that a series of compression balloons 620 of decreasing diameter can be used in sequence to incrementally reduce the diameter of the valve frame 50 until the valve frame 50 is sufficiently reduced in order to be removed from the implanted location.

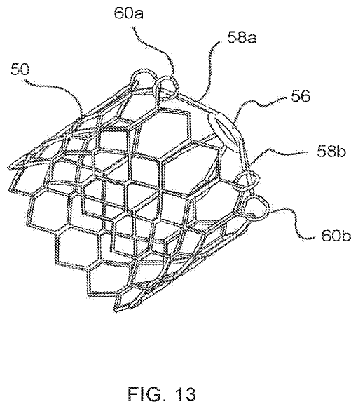

In FIGS. 11A and 11B, an alternate embodiment of the valve removal device 710 is shown comprised of a pre-shaped hook 726 with a distal end 728 that is configured to be positioned adjacent the hook shaft 727 when it is fully deployed from the hook containment tube 740. The pre-shaped hook 726 is kept inside the hook containment tube 740 until the hook containment tube 740 is delivered in proximity of the valve frame 50. Then the distal end 728 of the pre-shaped hook 726 is advanced out of the hook containment tube 740 and through the valve frame 50. Once the pre-shaped hook 726 has been completely advanced out of the hook containment tube 740 and through the valve frame 50, the distal end 728 of the pre-shaped hook 726 is positioned adjacent the hook shaft 727. The hook containment tube 740 can then be advanced over the distal end 728 of the pre-shaped hook 726 to lock the pre-shaped hook 726 to the valve frame 50. As with previous embodiments one or more of the pre-shaped hooks 726 and hook containment tubes 740 can be used to retrieve the valve frame 50 from its implant location and withdraw the valve frame 50 into the flexible shaft 20, or into a diameter reduction element 28 and then into the flexible shaft 20. The pre-shaped hook 726 can be formed from any material that has sufficient flexibility to be drawn substantially straight into a hook containment tube 720 and sufficient rigidity to form the required hook shape when it is deployed such as but not limited to a super elastic metal such as Nitinol (nickel titanium) or a flexible stainless steel.

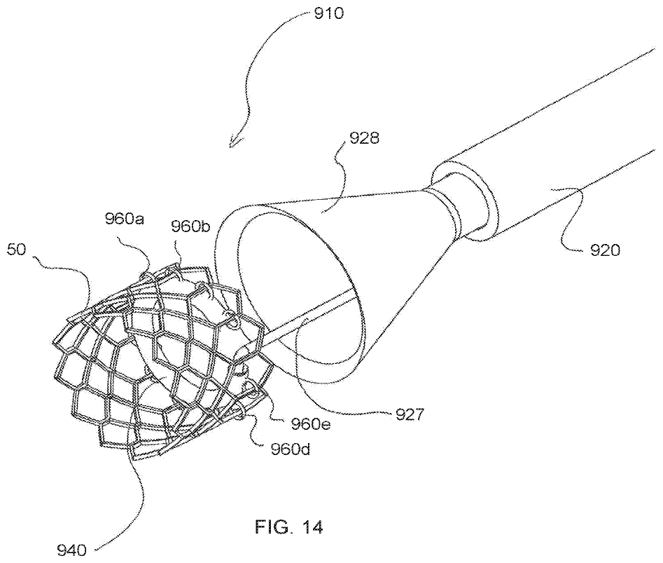

In FIGS. 12A and 12B, an alternate embodiment of the valve removal device 810 is shown comprised of a central shaft 827 and one or more engagement wires 826. The engagement wires 826 are fixed to the end of the central shaft 827 at the distal end and fixed to a wire hub 829 at the proximal end. The engagement wires 826 are straight against the central shaft 826 when the valve removal device 810 is first deployed into the patient's vasculature. Once positioned adjacent to the implanted valve frame 50, the central shaft 827 and wire hub 829 are advance out of the flexible shaft 820 and into the center of the valve frame 50. Then the central shaft 826 is retracted back towards the wire hub 829 which deploys the engagement wires 826 outwardly to the inner diameter of the valve frame 50. The inner diameter of the valve frame 50 is comprised of one or more wire docking extensions 854 that were formed in the valve frame 50 prior to implantation. The central shaft 826 and wire hub 829 are then rotated together to rotate the deployed engagement wires 826 until they engage the wire docking extensions 854. Then the central shaft 827 is extended forward through the valve frame 50 relative to the wire hub 829 which retracts the engagement wires 826 and reduces the diameter of the valve frame 50. The valve frame 50 can then be drawn into the flexible shaft 820, or into a diameter reduction element 828 and then into the flexible shaft 820. The proximal end of the engagement wire(s) 826 can be attached to the wire hub 829 as described or they can be attached directly to an engagement control lever 32 on the control handle 30.

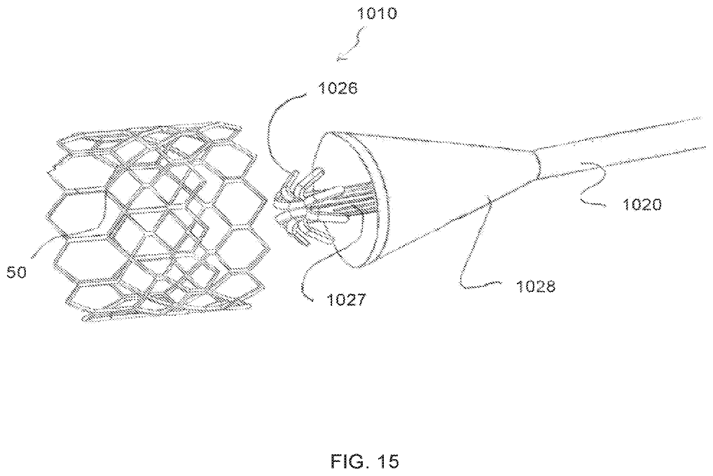

The valve frame 50 in FIGS. 12A and 12B had been modified by the addition of one or more wire docking extensions 854. There are many different modifications to a valve frame 50 that would likewise facilitate engagement with a valve removal device 10. One such modification is shown in FIG. 13, where several clips or rings 60a-b are attached to the valve frame 50. Each clip or ring 60a-b is attached to an extension 58a-b that connects the clips or rings 60a-b to a central dock 56. With this modification, any of the valve removal devices 10, 110, 210 310, 410, 510, 610, 710, 810, and 910 described herein with a single attachment element can be used to engage the central dock 56 and drawn the valve frame 50 into the flexible shaft 20, or into a diameter reduction element 28 and then into the flexible shaft 20. The need for only a single attachment element greatly simplifies the valve removal device 10 and simplifies the removal steps taken by the physician. The central dock 56 can be ring shaped as shown in FIG. 13 or it can take any number of shapes including but not limited to a knob, a hook, or a handle that facilitates engagement with a valve removal device 10.

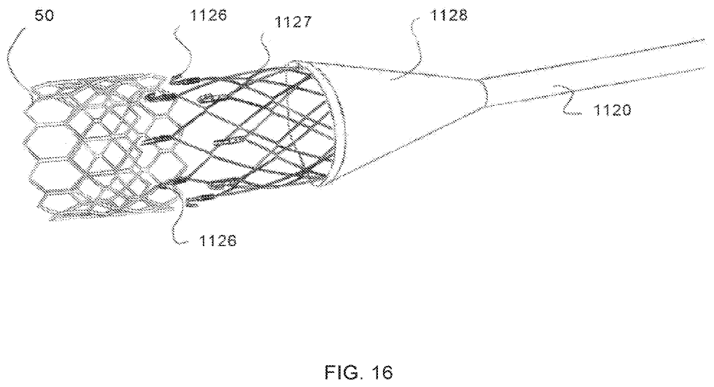

In FIG. 14, an alternate embodiment of the valve removal device 910 is shown comprised of an attachment tube 940, several attachment rings 960a-d and a tube collapsing element 927. The attachment tube 940 is connected to the valve frame 50 in multiple locations by the attachment rings 960a-d. The attachment rings 960a-d can be attached to the valve frame 50 by any number of means already described herein or well-known to those experts in the art, such as clipping, crimping, screwing, or welding. Once the attachment tube 940 is attached to the valve frame 50, the tube collapsing element 927 which is attached at the inside of the distal end of the attachment tube 940 is tensioned at the control handle 30 which collapses the attachment tube 940 on itself and reduces the diameter of the valve frame 50. Once the attachment tube 940 is fully collapsed, further tension on the tube collapsing element 927 draws the collapsed valve frame into the diameter reduction element 928 and then into the flexible shaft 920.

What is claimed is:

1. A method for removing a transcatheter heart valve from a heart in a patient, the method comprising:
providing a removal device comprising a control handle and a flexible shaft, the flexible shaft having a proximal end coupled to the control handle, a lumen, and a distal opening;
positioning the distal end of the flexible shaft such that the distal opening is disposed next to the transcatheter heart valve in the heart;
deploying one or more transcatheter engagement elements from the lumen through the distal opening of the flexible shaft such that the one or more engagement elements engage the transcatheter heart valve; and
retracting the one or more engagement elements such that the transcatheter heart valve is removed.

2. The method of claim 1, wherein the step of retracting the one or more engagement elements comprises pulling the transcatheter heart valve through the distal end into the lumen of the flexible shaft using the control handle.

3. The method of claim 1, further comprising deploying a diameter reduction element from the distal end of the flexible shaft, wherein the diameter reduction element reduces the diameter of the transcatheter heart valve when the one or more engagement elements pull the transcatheter heart valve through the distal end into the lumen of the flexible shaft.

4. The method of claim 3, wherein the diameter reduction element is a funnel.

5. The method of claim 1, wherein each engagement element comprises an elongate element that enables the engagement element to reach the transcatheter heart valve and a jaw that grips the transcatheter heart valve.

6. The method of claim 1, wherein each engagement element comprises an elongate element that enables the engagement element to reach the transcatheter heart valve and a locking gate that grips the transcatheter heart valve.

7. The method of claim 6, wherein the locking gate comprises a stop, a gate, and an articulating hinge.

8. The method of claim 1, wherein each engagement element comprises an engagement capture loop, a loop shaft, and an engagement capture wire.

9. The method of claim 8, wherein the engagement capture loop is advanced on one side of the transcatheter heart valve by extending the loop shaft, wherein the engagement capture wire is advanced on the other side of the transcatheter heart valve and inserted through the engagement capture loop, thereby capturing the transcatheter heart valve between the engagement capture loop and the engagement capture wire.

10. The method of claim 1, wherein each engagement element comprises an engagement coil.

11. The method of claim 10, wherein the engagement coil is tapered.

12. The method of claim 10, wherein the engagement coil comprises a variable pitch.

13. The method of claim 1, wherein the engagement element comprises a suture, a suture thrower, a suture grasping jaw, a jaw pivot, and a suture support shaft.

14. The method of claim 13, wherein the suture thrower advances the suture from one side of the transcatheter heart valve to the suture grasping jaw on the other side of the transcatheter heart valve.

15. The method of claim 13, wherein the jaw pivot enables the suture grasping jaw to open and close.

16. The method of claim 1, wherein each engagement element comprises a pre-shaped hook.

17. The method of claim 1, wherein each engagement element comprises a pre-shaped hook and a hook containment tube.

18. The method of claim 17, wherein the hook containment tube advances over the distal end of the pre-shaped hook to lock the pre-shaped hook to the transcatheter heart valve once the pre-shaped hook advances through the transcatheter heart valve.

19. The method of claim 1, wherein each engagement element comprises a self-expanding tube and an integrated engagement hook.

20. The device of claim 1, wherein each engagement element comprises a self-expanding tube and an attached engagement hook.

21. The method of claim 1, wherein the transcatheter heart valve comprises one or more wire docking extensions and the engagement element comprises a central shaft, one or more engagement wires, and a wire hub.

22. The method of claim 21, wherein the central shaft, the engagement wires, and the wire hub are rotated together to engage the wire docking extensions.

23. The method of claim 1, wherein the engagement element comprises an attachment tube, one or more attachment rings, and a tube collapsing element, wherein the attachment tube is connected to the transcatheter heart valve by the attachment rings.

24. The method of claim 1, wherein the transcatheter heart valve comprises one or more clips or rings, one or more extensions, and a central dock, wherein the clips or rings are attached to the central dock through the extensions.

25. The method of claim 1, wherein the removal device further comprises a compression balloon.

26. A method for removing a transcatheter heart valve from a heart in a patient, the method comprising:
providing a removal device comprising a control handle and a flexible shaft, the flexible shaft having a proximal end coupled to the control handle, a lumen, and a distal opening;
positioning the distal end of the flexible shaft such that the distal opening is disposed next to the transcatheter heart valve in the heart;
deploying a compression balloon from the lumen through the distal opening of the flexible shaft such that the compression balloon engages the transcatheter heart valve; and
retracting the one or more engagement elements such that the transcatheter heart valve is removed.

27. A removal device for removing a transcatheter heart valve from a heart in a patient, the removal device comprising:
a control handle and a flexible shaft, the flexible shaft having a proximal end coupled to the control handle, a lumen, and a distal opening; and
one or more transcatheter engagement elements disposed in the lumen and coupled to the control handle, the one or more engagement elements capable of being deployed through the distal opening of the flexible shaft such that the one or more engagement elements engage the transcatheter heart valve and remove the transcatheter heart valve.

28. The device of claim 27, further comprising a diameter reduction element which reduces the diameter of the transcatheter heart valve when the one or more engagement elements pull the transcatheter heart valve through the distal end into the lumen of the flexible shaft.

29. The device of claim 28, wherein the diameter reduction element comprises a wire mesh.

30. The device of claim 29, wherein the wire mesh is made from nitinol.

31. The device of any one of claims 28 to 30, wherein the diameter reduction element is a funnel.

32. The device of claim 27, wherein each engagement element comprises an elongate element that enables the engagement element to reach the transcatheter heart valve and a jaw that grips the transcatheter heart valve.

33. The device of claim 27, wherein each engagement element comprises an elongate element that enables the engagement element to reach the transcatheter heart valve and a locking gate that grips the transcatheter heart valve.

34. The device of claim 27, wherein each engagement element comprises an engagement capture loop, a loop shaft, and an engagement capture wire.

35. The device of claim 27, wherein each engagement element comprises an engagement coil.

36. The device of claim 27, wherein the engagement element comprises a suture, a suture thrower, a suture grasping jaw, a jaw pivot, and a suture support shaft.

37. The device of claim 27, wherein each engagement element comprises a pre-shaped hook.

38. The device of claim 27, wherein each engagement element comprises a pre-shaped hook and a hook containment tube.

39. The device of claim 27, wherein each engagement element comprises a self-expanding tube and an integrated engagement hook.

40. The device of claim 27, wherein each engagement element comprises a self-expanding tube and an attached engagement hook.

41. The device of claim 27, wherein the transcatheter heart valve comprises one or more wire docking extensions and the engagement element comprises a central shaft, one or more engagement wires, and a wire hub.

42. The device of claim 27, wherein the engagement element comprises an attachment tube, one or more attachment rings, and a tube collapsing element, wherein the attachment tube is connected to the transcatheter heart valve by the attachment rings.

43. The device of claim 27, wherein the removal device further comprises a compression balloon.
